(19) United States
(12) Patent Application Publication
(10) Pub. No.: US 2003/0171407 A1
(43) Pub. Date: Sep. 11, 2003

(54) Composition for reducing blood glucose and cholesterol

(75) Inventors: Lori M. Freese, Monticello, MN (US); Thomas R. Gorham, Brooklyn Park, MN (US); Jennifer A. Wheeler-Davis, Maple Grove, MN (US)

Correspondence Address: FAEGRE & BENSON LLP, 2200 WELLS FARGO CENTER, 90 SOUTH 7TH STREET, MINNEAPOLIS, MN, 55402, US

(73) Assignee: Upsher-Smith Laboratories, Inc.

(21) Appl. No.: 10/094,004

(22) Filed: Mar. 7, 2002

(51) Int. Cl. A61K 31/4439; A61K 31/426; A61K 31/366; A61K 31/401; A61K 31/225

(52) U.S. Cl. 514/342; 514/369; 514/423; 514/460; 514/548

(57) ABSTRACT

The invention provides a pharmaceutical composition which is a combination of an insulin-secretion stimulant and a HMG-CoA reductase inhibitor. Suitable insulin-secretion stimulants include the sulfonylurea drugs, and suitable HMG-CoA reductase inhibitors include the statin drugs. The composition may be formulated to provide extended-release characteristics of one or both of the active components. Also provided are methods for treating a diabetic patient using a combination of an insulin-secretion stimulant and a HMG-CoA reductase inhibitor. Practice of the methods of the invention may result in the administration of fewer dosages to the patient. The invention also provides a pharmaceutical composition which is a combination of an antihyperglycemic drug, particularly a biguanide compound, in combination with a HMG-CoA reductase inhibitor. Also provided are methods for treating a diabetic patient using a combination of an antihyperglycemic biguanide compound and a HMG-CoA reductase inhibitor.

### BACKGROUND

Diabetes is a disease characterized by elevated levels of blood plasma glucose, or hyperglycemia. Hyperglycemia, if uncontrolled, can lead to other complications, such as blindness, kidney disease, heart disease, stroke, nerve diseases, circulatory disorders, and impotence in males. Diabetes is a chronic disease with diverse pathologic manifestations and is accompanied by lipid metabolism and cardiovascular disorders as well as glycometabolism disorders.

Non-insulin dependent diabetes mellitus (NIDDM), or Type II diabetes, is characterized by either an inability to produce sufficient insulin, or an insensitivity to insulin. Type II diabetics are often prescribed blood glucose-lowering sulfonylurea-based or -derived drugs, which are associated with the stimulation of insulin production in the pancreatic β-cells. Alternatively, patients suffering from Type II diabetes may also be prescribed biguanide-based or -derived drugs, which are associated with increasing a patient's sensitivity to insulin.

Patients with diabetes are known to be at a greater risk of developing cardiovascular diseases including arteriosclerosis and atherosclerosis, due in part to a susceptibility to conditions such as hyperlipidemia or hypercholesterolemia. As a result, diabetic patients are recommended to maintain low levels of serum low-density lipoprotein (LDL) cholesterol. In particular, efforts are directed to maintaining a low serum LDL cholesterol level in a diabetic patient by regulation of diet and by therapeutic treatment using pharmaceutical agents.

A particularly effective class of pharmaceutical agents for reducing serum LDL cholesterol is the inhibitors of 3-hydroxy-3-methyl-glutaryl-coenzyme A (HMG-CoA) reductase. HMG-CoA reductase inhibitors generally act on the rate-limiting step of cholesterol biosynthesis, and thus act to decrease the amount of serum LDL cholesterol by reducing the quantity of total cholesterol produced within the body. The most widely used drugs of the HMG-CoA reductase class are statin drugs.

The treatment of diabetic patients using serum cholesterol-lowering statin drugs has been researched. U.S. Pat. No. 5,130,333 to Pan, et al. is directed to a method of reducing the risk of Type II diabetes by administering to a patient a cholesterol-lowering drug such as mevastatin, lovastatin, pravastatin or velostatin.

Various combination drugs or methods of treating patients using combination drugs have been developed. U.S. Pat. Nos. 5,798,375 and 6,159,997 to Tsujita, et al. are directed to methods of preventing or treating arteriosclerosis or xanthoma by administering to a patient a combination of a HMG-CoA reductase inhibitor and a thiazolidinedione insulin sensitizer. Also reported is a composition comprising a combination of a HMG-CoA reductase inhibitor and a thiazolidinedione insulin sensitizer. Preferred HMG-CoA reductase inhibitors include the statin drugs such as pravastatin, lovastatin, simvastatin, fluvastatin, rivastatin and atorvastatin.

U.S. Pat. Nos. 6,103,742, 6,121,295, and 6,169,100 to Ikeda, et al., are directed to methods of reducing the amount of active components administered to a patient, and reducing the side effects or complications associated with the administration of active components to diabetic patients. These methods include administering to the patient a combination of an insulin sensitivity enhancer and a statin. The reported insulin sensitivity enhancers (also known as insulin resistance deblockers) include pioglitazone, troglitazone and 5-[[4-[2(methyl-2-pyridylamino)ethoxyl]phenyl]-methyl]-2,4-thiazolidinedione.

U.S. Pat. Nos. 5,847,008 and 6,020,382 to Doebber, et al., and U.S. Pat. Nos. 5,859,051, 6,090,836 and 6,090,839 to Adams, et al. are directed to the treatment or prevention of diabetes or atherosclerosis by the administration of certain specified acetylphenol components. Also reported is the use of a combination of one of the specified acetylphenol components with either a sulfonylurea, fibrate, HMG-CoA reductase inhibitor, beta-sitosterol inhibitor, cholesterol acyltransferase inhibitor, biguanide, cholestyramine, angiotensin II antagonist, melinamide, nicotinic acid, fibrinogen receptor antagonist, aspirin, α-glucosidase inhibitor, insulin secretogogue or insulin, as well as other classes of drugs.

U.S. Pat. No. 6,160,000 to Adams, et al. is directed to pharmaceutical compositions for the prevention or treatment of diabetes and atherosclerosis, the compositions containing certain specified aryl and heteroaryl acetic acid and oxyacetic acid compounds. Also reported is the use of a combination of one of the specified aryl and heteroaryl acetic acid and oxyacetic acid compounds with either a sulfonylurea, fibrate, HMG-CoA reductase inhibitor, beta-sitosterol inhibitor, cholesterol acyltransferase inhibitor, biguanide, cholestyramine, angiotensin II antagonist, melinamide, nicotinic acid, fibrinogen receptor antagonist, aspirin, α-glucosidase inhibitor, insulin secretogogue or insulin, as well as other classes of drugs.

### SUMMARY OF THE INVENTION

In one embodiment, the present invention is a novel pharmaceutical composition for treating diabetic patients. The composition is a combination of a blood glucose-lowering drug and a serum cholesterol-lowering drug in a single pharmaceutical dosage unit. In particular, the novel composition is a combination of an insulin-secretion stimulant and a HMG-CoA reductase inhibitor. In some embodiments, one or both of the drugs exhibits sustained-release properties.

A particular pharmaceutical dosage unit containing both glipizide and simvastatin is also provided by the present invention. The dosage unit may be formulated so that one or both of the glipizide and simvastatin exhibit sustained-release properties.

In another embodiment, the present invention is a novel pharmaceutical composition for treating diabetic patients. The composition is a combination of an antihyperglycemic drug and a serum cholesterol-lowering drug in a single pharmaceutical dosage unit. In particular, the inventive composition is a combination of a biguanide compound and a HMG-CoA reductase inhibitor. In some embodiments, one or both of the drugs exhibits sustained-release properties.

In another embodiment, the present invention is a method of treating a diabetic patient by administering a combination of a blood glucose-lowering drug and a serum cholesterol-lowering drug in a single pharmaceutical dosage unit. In particular, the dosage unit may contain a combination of an insulin-secretion stimulant and a HMG-CoA reductase inhibitor. In some embodiments, one or both of the drugs exhibits sustained-release properties. Also provided is a method of treating a diabetic patient by administering a combination of an antihyperglycemic biguanide compound and a serum cholesterol-lowering drug in a single pharmaceutical dosage unit. Suitable serum cholesterol-lowering drugs include the HMG-CoA reductase inhibitors. In some embodiments, one or both of the drugs exhibits sustained-release properties.

The present invention also provides a method for reducing the number of pharmaceutical dosages that is required to be administered to a diabetic patient. The method comprises the steps of combining in a single pharmaceutical dosage unit both an insulin-secretion stimulant and a HMG-CoA reductase inhibitor, and administering the dosage unit to a diabetic patient. Also provided is a method for reducing the number of pharmaceutical dosages that is required to be administered to a diabetic patient, the method comprising the steps of combining in a single pharmaceutical dosage unit both an antihyperglycemic biguanide compound and a HMG-CoA reductase inhibitor, and administering the dosage unit to a diabetic patient.

Further, the present invention provides a method in which a diabetic patient can be administered a single dosage unit per day, the dosage unit including both an insulin-secretion stimulant and a HMG-CoA reductase inhibitor. Likewise, the present invention provides a method in which a diabetic patient can be administered a single dosage unit per day, the dosage unit including both an antihyperglycemic biguanide compound and a HMG-CoA reductase inhibitor.

### DETAILED DESCRIPTION OF THE INVENTION

In one embodiment, the present invention is a novel pharmaceutical composition for treating diabetic patients. The novel composition is a combination of a blood glucose-lowering compound and a serum cholesterol-lowering compound in a single pharmaceutical dosage unit. In particular, the novel composition is a combination of an insulin-secretion stimulant and a HMG-CoA reductase inhibitor.

One suitable class of blood glucose-lowering compounds is the sulfonylurea-based or -derived drugs, referred to collectively herein as “sulfonylurea drugs.” The primary mode of action of sulfonylurea drugs is by stimulation of insulin secretion from pancreatic β-cells. Sulfonylurea-based or -derived pharmaceutical compounds presently known include the so-called “second generation” sulfonylurea drugs glipizide, glimepiride, and glyburide. Second-generation sulfonylurea drugs are presently marketed under the trade names GLUCOTROL™ and GLUCOTROL XL™ (glipizide), AMARYL™ (glimepiride), and MICRONASE™, GLYNASE PresTab™ and Diaβeta™ (glyburide). The most preferred glucose-lowering drug in the sulfonylurea class for the practice of the present invention is glipizide. Alternative sulfonylurea drugs include the so-called “first-generation” sulfonylurea drugs tolbutamide, chlorpropamide, tolazamide, and acetohexamide.

The preferred group of serum cholesterol-lowering compounds is the inhibitors of 3-hydroxy-3-methyl-glutaryl-coenzyme A (HMG-CoA) reductase. In the description and claims of the present invention, the term “HMG-CoA reductase inhibitor” is intended to include compounds that are active as HMG-CoA reductase inhibitors in an administered form, as well as compounds that are inactive in administered form but are hydrolyzed or otherwise modified in vivo to yield an active HMG-CoA reductase inhibitor.

Pharmaceutical agents presently known in the HMG-CoA reductase inhibitor group include the statin drugs. Presently known statin drugs include simvastatin, atorvastatin calcium, fluvastatin sodium, lovastatin, pravastatin sodium, and rosuvastatin calcium. HMG-CoA reductase inhibitor drugs are presently marketed under the trade names ZOCOR™ (simvastatin), LIPITOR™ (atorvastatin calcium), LESCOL™ (fluvastatin sodium), MEVACOR™ (lovastatin), and PRAVACHOL™ (pravastatin sodium). CRESTOR™ (rosuvastatin calcium) is presently under regulatory review. A suitable statin for the practice of the present invention is simvastatin.

A particular combination of a blood glucose-lowering drug and a serum cholesterol-lowering drug for the practice of the present invention is the combination of glipizide and simvastatin in a single pharmaceutical dosage unit. A particular dosage unit, comprising about 5 to about 10 milligrams glipizide and about 20 to about 40 milligrams simvastatin, is also provided by the present invention.

Other suitable combinations in the practice of the present invention include: simvastatin and glimepiride; simvastatin and glyburide; atorvastatin calcium and glipizide; atorvastatin calcium and glimepiride; atorvastatin calcium and glyburide; fluvastatin sodium and glipizide; fluvastatin sodium and glimepiride; fluvastatin sodium and glyburide; lovastatin and glipizide; lovastatin and glimepiride; lovastatin and glyburide; pravastatin sodium and glipizide; pravastatin sodium and glimepiride; pravastatin sodium and glyburide; rosuvastatin calcium and glipizide; rosuvastatin calcium and glimepiride; rosuvastatin calcium and glyburide.

The pharmaceutical dosage units of the present invention may be formulated for any form of administration, including, for example, oral administration as tablets or capsules. Presently, a tablet is the preferred dosage unit for oral administration. The necessary ingredients for the dosage unit may be processed in accordance with known methods, using or incorporating familiar coatings and additives as required. By way of example, in addition to the pharmaceutically active components, a dosage unit may contain binders, fillers, disintegrants, sustained-release agents, diluents, antiadherents, glidants, flow aids, plasticizers and lubricants, which are well-known in the field of pharmaceutical processing.

In specific embodiments of the inventive combination, the pharmaceutical dosage unit is formulated so that one or both of the active components (i.e., the blood glucose-lowering drug and the serum cholesterol-lowering drug) exhibits sustained-release characteristics upon administration to the patient. In particular, the dosage unit may be formulated for sustained release of the blood glucose-lowering component, thereby enabling the administration of a single dosage unit per day. By way of example, a glipizide/simvastatin combination dosage unit would most preferably be formulated for sustained release of glipizide, thereby enabling once-daily administration of the dosage unit.

As used in this specification and in the claims, the phrase “sustained release” indicates that an active component is released from the dosage unit over a period of time which is longer than its ordinary in vivo half-life, thus extending the presence of the component in a patient's system considerably beyond its ordinary half-life. Under such circumstances, the active component is said to “exhibit sustained-release characteristics.” In contrast, the phrase “immediate release” indicates that an active component is released from the dosage unit within a short period of time (i.e., much shorter than its ordinary in vivo half-life); and decrease in concentration of the active component over time will be approximately described by its ordinary half-life. Under such circumstances, the active component is said to “exhibit immediate-release characteristics.” In compositions of the present invention having more than one active component that is intended to exhibit sustained-release characteristics, an important consideration is that the release rate of each active component will need to be separately customized to produce the desired release profile, since the components will have different in vivo half-lives.

In accordance with the present invention, a tablet-form dosage unit having a hydrophilic hydroxypropyl methylcellulose (HPMC) matrix may be formulated which exhibits sustained release characteristics for at least one of the active components. In addition to the active components, this type of tablet includes high-viscosity hydroxypropyl methylcellulose as a sustained-release agent, microcrystalline cellulose and starch as bulking agents, a hydroxypropyl methylcellulose binder, talc as an antiadherent and glidant, and magnesium stearate as a lubricant. Using conventional processes, the ingredients are combined and pressed into tablets.

Hydroxypropyl methylcellulose, as provided by Dow Chemical (Midland, Mich.) under the trade name METHOCEL, is available in several different grades having varying ratios of hydroxypropyl and methyl substitution, a factor which influences organic solubility and the thermal gelation temperature of aqueous solutions. Suitable products for use as the high-viscosity HPMC component in the various embodiments of this invention include the METHOCEL K Premium Series, for example, METHOCEL K 4M, METHOCEL K 15M, or METHOCEL K 100M. A second HPMC component having lower viscosity may be used as a binder or as a film former. Suitable products for use as a binder component or film former include the METHOCEL E Premium Series, such as METHOCEL E 5 or METHOCEL E 15.

When the tablet of this embodiment encounters an aqueous environment, such as in the digestive tract, the outer portion of the tablet partially hydrates and a gel layer is formed. The type and amount of high-viscosity HPMC sustained-release agent controls the rate of diffusion of the active ingredients into the aqueous environment and the rate of erosion of the tablet. For water-insoluble active ingredients useful in compositions of the present invention such as, for example, simvastatin, lovastatin, glipizide, and glyburide, tablet erosion is the primary mechanism responsible for the release rate of the active ingredient. For water-soluble active ingredients useful in compositions of the present invention such as, for example, fluvastatin sodium, rosuvastatin calcium or pravastatin sodium, diffusion of the active ingredient into the aqueous environment may be the dominating factor that determines the release rate of the active ingredient.

A particular formulation in accordance with the above-described embodiment of the present invention comprises the following components, given by wt.-% of the dosage unit: glipizide, 3.3 wt.-%; simvastatin, 13.3 wt.-%; microcrystalline cellulose and starch bulking agents, 65.9 wt.-%; high-viscosity HPMC sustained-release agent, 8.0 wt.-%; HPMC binder, 5.0 wt.-%; talc glidant/antiadherent, 4.0 wt.-%; magnesium stearate lubricant, 0.5 wt.-%. Using conventional processes, the ingredients are combined and pressed into tablets prior to administration to a patient.

Also in accordance with the present invention, tablet- or capsule-form dosage units comprising ethylcellulose-coated particles of at least one of the active components may be made. In the practice of this embodiment, particles of at least one active component are first coated with ethylcellulose, and in some cases, with a pore former such as HPMC and a plasticizer such as dibutyl sebacate, using conventional processes. For use in capsules, the coated particles are then mixed with bulking agents such as lactose and microcrystalline cellulose. Capsules are then filled with an appropriate amount of this mixture. For use in tablets, the coated particles are mixed with lactose and microcrystalline cellulose bulking agents, croscarmellose sodium as a disintegrant, silicon dioxide as an antiadherent, and magnesium stearate as a lubricant. This mixture may then be pressed into tablets.

A suitable ethylcellulose material is provided by Dow Chemical (Midland, Mich.) under the trade name ETHOCEL. In particular, suitable ethylcellulose materials useful for controlled-release coating include ETHOCEL Standard 7 Premium, ETHOCEL Standard 10 Premium, and ETHOCEL Standard 20 Premium. A suitable HPMC material for use as a pore former is METHOCEL E5.

When the ethylcellulose-coated particles of either the tablet or capsule of this embodiment encounter an aqueous environment, such as in the digestive tract, the coated particles are dispersed into the aqueous environment when the other soluble ingredients dissolve. Drug release is by diffusion through the ethylcellulose coating. The rate of drug release is dependent upon the percentage of ethylcellulose used in the dosage unit, the amount of coating applied to the particles of active components, and the presence and amount of any film former applied.

A particular capsule-form formulation in accordance with the above-described embodiment of the present invention comprises the following components, given by wt.-% of the capsule filler: glipizide, 3.3 wt.-%; simvastatin, 13.3 wt.-%; microcrystalline cellulose and lactose bulking agents, 69.4 wt.-%; ethylcellulose sustained-release agent, 14.0 wt.-%. Using conventional processes, the active components are coated with ethylcellulose, the coated particles are mixed with the other ingredients, and capsules are filled with the mixture prior to administration of the filled capsule to a patient.

A particular tablet-form formulation in accordance with the above-described embodiment of the present invention comprises the following components, given by wt.-% of the dosage unit: glipizide, 3.3 wt.-%; simvastatin, 13.3 wt.-%; microcrystalline cellulose and lactose bulking agents, 67.7 wt.-%; ethylcellulose sustained-release agent, 14.0 wt.-%; croscarmellose sodium disintegrant, 1.0 wt.-%; silicon dioxide antiadherent, 0.5 wt.-%; magnesium stearate lubricant, 0.2 wt.-%. Using conventional processes, the active components are coated with ethylcellulose, and the coated particles and other ingredients are combined and pressed into tablets prior to administration of the tablet to a patient.

Also in accordance with the present invention, tablet- or capsule-form dosage units comprising the active components in an acrylic polymer matrix may be made. Preferred acrylic polymers include methacrylic acid copolymers and ammoniomethacrylate copolymers. In the practice of this embodiment, particles of the active component may first optionally be coated with an acrylic polymer and a plasticizer such as dibutyl sebacate. For use in capsules, the coated particles are then mixed with bulking agents such as microcrystalline cellulose. Capsules are then filled with an appropriate amount of the mixture. Alternatively, the acrylic polymer and active components may be mixed with other ingredients and compressed. For use in tablets, the active components and acrylic polymer may be mixed with dicalcium phosphate and starch bulking agents, talc as an glidant/antiadherent, and stearic acid as a lubricant. The mixture may then be pressed into tablets.

A particular methacrylic acid copolymer that may be used in the acrylic polymer matrix is EUDRAGIT L100, provided by Rohm America (Piscataway, N.J.). A particular ammoniomethacrylate copolymer that may be used in the acrylic polymer matrix is EUDRAGIT RS100, provided by Rohm America.

When the tablet or capsule of this embodiment encounters an aqueous environment, such as in the digestive tract, the acrylic polymer matrix structure remains intact. Drug release is by diffusion through the polymer matrix. The rate of drug release is dependent upon the percentage of acrylic polymer in the dosage unit, or by the amount of acrylic polymer coating applied to particles of active components.

A particular capsule-form formulation in accordance with the above-described embodiment of the present invention comprises the following components, given by wt.-% of the capsule filler: glipizide, 3.3 wt.-%; simvastatin, 13.3 wt.-%; microcrystalline cellulose bulking agent, 64.6 wt.-%; methacrylic acid copolymer sustained-release agent, 18.0 wt.-%; dibutyl sebacate plasticizer, 0.8 wt.-%.

A particular tablet-form formulation in accordance with the above-described embodiment of the present invention comprises the following components, given by wt.-% of the dosage unit: glipizide, 3.3 wt.-%; simvastatin, 13.3 wt.-%; dicalcium phosphate and starch bulking agents, 63.6 wt.-%; ammoniomethacrylate copolymer sustained-release agent, 18.0 wt.-%; talc glidant/antiadherent, 1.0 wt.-%; stearic acid lubricant, 0.8 wt.-%.

Also in accordance with the present invention, a tablet-form dosage unit having a partially hydrophilic matrix may be made which exhibits sustained release of at least one of the active components. In addition to the active components, the tablet is comprised of ethylcellulose as a sustained-release agent and HPMC as a film former, in addition to microcrystalline cellulose and starch as bulking agents, a polyvinylpyrrolidone binder, silicon dioxide as an antiadherent, dibutyl sebacate as a plasticizer, and magnesium stearate as a lubricant. Suitable HPMC and ethylcellulose materials are listed above. Using conventional processes, the listed ingredients, other than ethylcellulose, HPMC and dibutyl sebacate, are combined and pressed into a tablet. The tablet is then coated with the ethylcellulose, HPMC and dibutyl sebacate prior to administration of the tablet.

When this tablet encounters an aqueous environment, such as in the digestive tract, the water-soluble portions of the tablet coating dissolve, leaving a non-continuous film of water-insoluble ethylcellulose surrounding the remaining tablet core. The rate of diffusion of the active ingredients from the tablet core into the aqueous environment is determined by the concentration of ethylcellulose, HPMC and dibutyl sebacate in the coating.

A particular formulation in accordance with the above-described embodiment of the present invention comprises the following components, given by wt.-% of the dosage unit: glipizide, 3.3 wt.-%; simvastatin, 13.3 wt.-%; microcrystalline cellulose and starch bulking agents, 72.5 wt.-%; polyvinylpyrrolidone binder, 4.0 wt.-%; silicon dioxide antiadherent, 0.5 wt.-%; magnesium stearate lubricant, 0.2 wt.-%. Using conventional processes, the ingredients are combined and pressed into tablets, and are coated prior to administration of the tablet with a mixture of: ethylcellulose sustained release agent, 4.0 wt.-% of the dosage unit; HPMC film former, 2.0 wt.-%; and dibutyl sebacate plasticizer, 0.2 wt.-%.

In some embodiments of the present invention, the pharmaceutical dosage unit is formulated so that a serum cholesterol-lowering drug exhibits immediate-release characteristics upon administration to the patient. In these embodiments, it is still preferred that the dosage unit is formulated for sustained release of the blood glucose-lowering component, thereby enabling the administration of a single dosage unit per day.

By way of demonstration, the above-described advantage may be realized for tablet-form dosage units by, for example, creating a bi-layer tablet, having one layer formulated as described by the above formulations to provide sustained release of the blood glucose-lowering component, and a second layer formulated to provide immediate release of a serum cholesterol-lowering component. Formation of bi-layer tablets is disclosed in U.S. Pat. Nos. 4,786,503 and 4,946,685 to Edgren, et al. and the references cited therein.

For capsule-form dosage units, the above-described advantage may be realized by, for example, including in the capsule the filler described by the above formulations to provide sustained release of the blood glucose-lowering component, and a second fill material providing immediate release of a serum cholesterol-lowering component.

Another aspect of the present invention is a novel method of treating a diabetic patient, the method comprising administering a pharmaceutical dosage unit including a combination of an insulin-secretion stimulant and a HMG-CoA reductase inhibitor. In the practice of this method, any of the above-described pharmaceutical compositions is suitable as the pharmaceutical dosage unit.

Yet another aspect of the present invention is a novel method of treating a diabetic patient, the method comprising administering a once-daily pharmaceutical dosage unit comprising a combination of an insulin-secretion stimulant and a HMG-CoA reductase inhibitor. In the practice of this method, upon administration to the patient the pharmaceutical dosage unit exhibits sustained-release properties as to the insulin-secretion stimulant, so that the patient does not require any further dosages throughout the day. Any of the above-described pharmaceutical compositions having sustained-release properties for the insulin-secretion stimulant and providing enough insulin-secretion stimulant for an entire day's treatment is suitable as the pharmaceutical dosage unit in the practice of this method of the present invention. As a result of the practice of this method, a single dosage per day may be administered to a diabetic patient who would otherwise require multiple dosages per day.

The present invention also provides a method for reducing the number of dosages administered to a diabetic patient by utilizing a pharmaceutical dosage unit comprising a combination of active agents. The method comprises the steps of combining in a single dosage unit a therapeutically effective amount of an insulin-secretion stimulant and a HMG-CoA reductase inhibitor, and administering to a diabetic patient the pharmaceutical dosage unit. In the practice of this method, any of the above-described pharmaceutical compositions is suitable as the pharmaceutical dosage unit. As a result of the practice of this method, a single dosage may be administered to a diabetic patient, the single dosage replacing multiple dosages that would otherwise be administered.

The appropriate dosages for administration to a patient for any of the compositions or methods of the present invention should be determined in accordance with accepted guidelines, such as those given in the Physician's Desk Reference. Drugs used for the control and management of Type II diabetes are not administered by a fixed dosage regimen. Rather, the patient's response to the drug must be observed (such as by measuring blood glucose or other indicators) and the dosage adjusted accordingly. The term “therapeutically effective amount” in this specification and in the claims is used to indicate a dosage that is effective in, or is targeted to, maintaining a desired level of blood glucose over an appropriate time window. For informational purposes only, the recommended daily dosage range for glipizide is in the range 2.5-40 milligrams/day; for glyburide, 2.5-20 milligrams/day; for glimepiride, 1-8 milligrams/day.

The administration of serum cholesterol-lowering drugs is less sensitive, but should also be determined in accordance with accepted guidelines, such as those given in the Physician's Desk Reference. For informational purposes only, the recommended daily dosage range for simvastatin is in the range 5-80 milligrams/day; for atorvastatin calcium, 10-80 milligrams/day; for fluvastatin sodium, 20-80 milligrams/day; for lovastatin, 10-80 milligrams/day; and for pravastatin sodium, 10-40 milligrams/day. Dosage guidelines for rosuvastatin calcium are expected to be in the range 5-80 milligrams/day.

In yet another aspect, the present invention provides another pharmaceutical composition for treating diabetic patients. The novel composition is a combination of an antihyperglycemic drug and a serum cholesterol-lowering drug. The serum cholesterol-lowering drugs suitable for use in this composition of the present invention are the HMG-CoA reductase inhibitors described above. In one embodiment, the serum cholesterol-lowering drug is a statin, such as, for example, simvastatin. A class of antihyperglycemic drugs for use in the compositions of these embodiments of the present invention is the biguanide-based or -derived compounds, referred to collectively herein as “biguanide compounds.” Pharmaceutically active agents presently known as antihyperglycemic biguanide compounds include phenformin, buformin and metformin hydrochloride. Metformin hydrochloride is presently marketed under the trade name GLUCOPHAGE™. Metformin hydrochloride is a suitable antihyperglycemic drug for the practice of an embodiment of the present invention.

An example of a combination of an antihyperglycemic drug and a serum cholesterol-lowering drug for the practice of an embodiment of the present invention is the combination of metformin hydrochloride and simvastatin in a single pharmaceutical dosage unit. In certain embodiments, the pharmaceutical dosage unit is formulated so that one or both of the active components (i.e., the antihyperglycemic drug and the serum cholesterol-lowering drug) exhibits sustained-release characteristics upon administration to the patient. The dosage unit may also be formulated so that the HMG-CoA reductase inhibitor exhibits immediate-release characteristics upon administration to the patient.

Other suitable combinations include metformin hydrochloride and atorvastatin calcium; metformin hydrochloride and fluvastatin sodium; metformin hydrochloride and lovastatin; metformin hydrochloride and pravastatin sodium; metformin hydrochloride and rosuvastatin calcium.

In accordance with the present invention, a tablet-form dosage unit having a hydrophilic hydroxypropyl methylcellulose (HPMC) matrix may be formulated which exhibits sustained release characteristics for at least one of the active components. In addition to the active components, this type of tablet includes high-viscosity hydroxypropyl methylcellulose as a sustained-release agent, in addition to microcrystalline cellulose and starch as bulking agents, a hydroxypropyl methylcellulose binder, talc as an antiadherent and glidant, and magnesium stearate as a lubricant. Suitable HPMC materials for the practice of this embodiment are listed above. Using conventional processes, the ingredients are combined and pressed into tablets.

When this tablet encounters an aqueous environment, such as in the digestive tract, the outer portion of the tablet partially hydrates and a gel layer is formed. The type and amount of HPMC sustained-release agent controls the rate of diffusion of the active ingredients into the aqueous environment and the rate of erosion of the tablet. For water-insoluble active ingredients useful in compositions of the present invention such as, for example, simvastatin or lovastatin, tablet erosion is the primary mechanism responsible for the release rate of the active ingredient. For water-soluble active ingredients useful in compositions of the present invention such as, for example, fluvastatin sodium, rosuvastatin calcium, pravastatin sodium or metformin hydrochloride, diffusion of the active ingredient into the aqueous environment may be the dominating factor that determines the release rate of the active ingredient.

A particular formulation in accordance with the above-described embodiment of the present invention comprises the following components, given by wt.-% of the dosage unit: metformin hydrochloride, 75.0 wt.-%; simvastatin, 1.0 wt.-%; microcrystalline cellulose and starch bulking agents, 6.5 wt.-%; high-viscosity HPMC sustained-release agent, 8.0 wt.-%; HPMC binder, 5.0 wt.-%; talc glidant/antiadherent, 4.0 wt.-%; magnesium stearate lubricant, 0.5 wt.-%. Using conventional processes, the ingredients are combined and pressed into tablets prior to administration to a patient.

Also in accordance with the present invention, tablet- or capsule-form dosage units comprising ethylcellulose-coated particles of at least one of the active components may be made. In the practice of this embodiment, particles of at least one active component are first coated with ethylcellulose, and in some cases, with a pore former such as HPMC and a plasticizer such as dibutyl sebacate, using conventional processes. Suitable HPMC and ethylcellulose materials are listed above. For use in capsules, the coated particles are then mixed with bulking agents such as lactose and microcrystalline cellulose. Capsules are then filled with an appropriate amount of this mixture. For use in tablets, the coated particles are mixed with lactose and microcrystalline cellulose bulking agents, croscarmellose sodium as a disintegrant, silicon dioxide as an antiadherent, and magnesium stearate as a lubricant. This mixture may then be pressed into tablets.

When the ethylcellulose-coated particles of either the tablet or capsule of this embodiment encounter an aqueous environment, such as in the digestive tract, the coated particles are dispersed into the aqueous environment when the other soluble ingredients dissolve. Drug release is by diffusion through the ethylcellulose coating. The rate of drug release is dependent upon the percentage of ethylcellulose used in the dosage unit, the amount of coating applied to the particles of active components, and the presence and amount of film former applied.

A particular capsule-form formulation in accordance with the above-described embodiment of the present invention comprises the following components, given by wt.-% of the capsule filler: metformin hydrochloride, 75.0 wt.-%; simvastatin, 1.0 wt.-%; microcrystalline cellulose and lactose bulking agents, 10.0 wt.-%; ethylcellulose sustained-release agent, 14.0 wt.-%. Using conventional processes, the active components are coated with ethylcellulose, the coated particles are mixed with the other ingredients, and capsules are filled with the mixture prior to administration of the filled capsule to a patient.

A particular tablet-form formulation in accordance with the above-described embodiment of the present invention comprises the following components, given by wt.-% of the dosage unit: metformin hydrochloride, 75.0 wt.-%; simvastatin, 1.0 wt.-%; microcrystalline cellulose and lactose bulking agents, 8.3 wt.-%; ethylcellulose sustained-release agent, 14.0 wt.-%; croscarmellose sodium disintegrant, 1.0 wt.-%; silicon dioxide antiadherent, 0.5 wt.-%; magnesium stearate lubricant, 0.2 wt.-%. Using conventional processes, the active components are coated with ethylcellulose, and the coated particles and other ingredients are combined and pressed into tablets prior to administration of the tablet to a patient.

Also in accordance with the present invention, tablet- or capsule-form dosage units comprising the active components in an acrylic polymer matrix may be made. Preferred acrylic polymers include methacrylic acid copolymers and ammoniomethacrylate copolymers. Suitable methacrylic acid copolymers and ammoniomethacrylate copolymers are listed above. In the practice of this embodiment, particles of the active component may first optionally be coated with an acrylic polymer and a plasticizer such as dibutyl sebacate. For use in capsules, the coated particles are then mixed with bulking agents such as microcrystalline cellulose. Capsules are then filled with an appropriate amount of the mixture. Alternatively, the acrylic polymer and active components may be mixed with other ingredients and compressed. For use in tablets, the active components and acrylic polymer may be mixed with dicalcium phosphate and starch bulking agents, talc as an glidant/antiadherent, and stearic acid as a lubricant. The mixture may then be pressed into tablets.

When the tablet or capsule of this embodiment encounters an aqueous environment, such as in the digestive tract, the acrylic polymer matrix structure remains intact. Drug release is by diffusion through the polymer matrix. The rate of drug release is dependent upon the percentage of acrylic polymer in the dosage unit, or by the amount of acrylic polymer coating applied to particles of active components.

A particular capsule-form formulation in accordance with the above-described embodiment of the present invention comprises the following components, given by wt.-% of the capsule filler: metformin hydrochloride, 75.0 wt.-%; simvastatin, 1.0 wt.-%; microcrystalline cellulose bulking agent, 5.2 wt.-%; methacrylic acid copolymer sustained-release agent, 18.0 wt.-%; dibutyl sebacate plasticizer, 0.8 wt.-%.

A particular tablet-form formulation in accordance with the above-described embodiment of the present invention comprises the following components, given by wt.-% of the dosage unit: metformin hydrochloride, 75.0 wt.-%; simvastatin, 1.0 wt.-%; dicalcium phosphate and starch bulking agents, 4.2 wt.-%; ammoniomethacrylate copolymer sustained-release agent, 18.0 wt.-%; talc glidant/antiadherent, 1.0 wt.-%; stearic acid lubricant, 0.8 wt.-%.

Also in accordance with the present invention, a tablet-form dosage unit having a partially hydrophilic matrix may be made which exhibits sustained release of at least one of the active components. In addition to the active components, the tablet is comprised of ethylcellulose as a sustained-release agent and HPMC as a film former, in addition to microcrystalline cellulose and starch as bulking agents, a polyvinylpyrrolidone binder, silicon dioxide as an antiadherent, dibutyl sebacate as a plasticizer, and magnesium stearate as a lubricant. Suitable HPMC and ethylcellulose materials are listed above. Using conventional processes, the ingredients, other than ethylcellulose, HPMC and dibutyl sebacate, are combined and pressed into a tablet. The tablet is then coated with the ethylcellulose, HPMC and dibutyl sebacate prior to administration of the tablet.

When this tablet encounters an aqueous environment, such as in the digestive tract, the water-soluble portions of the tablet coating dissolve, leaving a non-continuous film of water-insoluble ethylcellulose surrounding the remaining tablet core. The rate of diffusion of the active ingredients from the tablet core into the aqueous environment is determined by the concentration of ethylcellulose, HPMC and dibutyl sebacate in the coating.

A particular formulation in accordance with the above-described embodiment of the present invention comprises the following components, given by wt.-% of the dosage unit: metformin hydrochloride, 75.0 wt.-%; simvastatin, 1.0 wt.-%; microcrystalline cellulose and starch bulking agents, 13.1 wt.-%; polyvinylpyrrolidone binder, 4.0 wt.-%; silicon dioxide antiadherent, 0.5 wt.-%; magnesium stearate lubricant, 0.2 wt.-%. Using conventional processes, the ingredients are combined and pressed into tablets, and are coated prior to administration of the tablet with a mixture of: ethylcellulose sustained release agent, 4.0 wt.-% of the dosage unit; HPMC film former, 2.0 wt.-%; and dibutyl sebacate plasticizer, 0.2 wt.-%.

Another aspect of the present invention is a novel method of treating a diabetic patient, the method comprising administering a pharmaceutical dosage unit including a combination of an antihyperglycemic biguanide compound and a HMG-CoA reductase inhibitor. In the practice of this method, any of the above-described pharmaceutical compositions comprising an antihyperglycemic biguanide compound is suitable as the pharmaceutical dosage unit.

Another aspect of the present invention is a novel method of treating a diabetic patient, the method comprising administering a once-daily pharmaceutical dosage unit comprising a combination of an antihyperglycemic biguanide compound and a HMG-CoA reductase inhibitor. In the practice of this method, upon administration to the patient the pharmaceutical dosage unit exhibits sustained-release properties as to the antihyperglycemic biguanide compound, so that the patient does not require any further dosages throughout the day. Any of the above-described pharmaceutical compositions having sustained-release properties for the antihyperglycemic biguanide compound and providing enough antihyperglycemic biguanide compound for an entire day's treatment is suitable as the pharmaceutical dosage unit in the practice of this method of the present invention. As a result of the practice of this method, a single dosage per day may be administered to a diabetic patient who would otherwise require multiple dosages per day.

The present invention also provides a method for reducing the number of dosages administered to a diabetic patient by utilizing a pharmaceutical dosage unit comprising a combination of active agents. The method comprises the steps of combining in a single dosage unit a therapeutically effective amount of an antihyperglycemic biguanide compound and a HMG-CoA reductase inhibitor, and administering to a diabetic patient the pharmaceutical dosage unit. In the practice of this method, any of the above-described pharmaceutical compositions comprising an antihyperglycemic biguanide compound is suitable as the pharmaceutical dosage unit. As a result of the practice of this method, a single dosage may be administered to a diabetic patient, the single dosage replacing multiple dosages that would otherwise be administered.

The appropriate dosages for administration to a patient for any of the compositions or methods of the present invention should be determined in accordance with accepted guidelines, such as those given in the Physician's Desk Reference. Drugs used for the control and management of Type II diabetes are not administered by a fixed dosage regimen. Rather, the patient's response to the drug must be observed (such as by measuring blood glucose or other indicators) and the dosage adjusted accordingly. The term “therapeutically effective amount” in this specification and in the claims is used to indicate a dosage that is effective in, or is targeted to, maintaining a desired level of blood glucose over an appropriate time window. For informational purposes only, the recommended daily dosage range for metformin hydrochloride is in the range 1500-2550 milligrams/day.

This invention, as set out in the appended claims, is not to be taken as limited to all of the details set out in this specification, as modifications and variations thereof may be made without departing from the spirit or scope of the invention.

What is claimed is:

1. A pharmaceutical dosage unit comprising a therapeutically effective amount of an insulin secretion stimulant in combination with a HMG-CoA reductase inhibitor.

2. The dosage unit of claim 1, wherein the insulin secretion stimulant is a sulfonylurea drug.

3. The dosage unit of claim 1, wherein the HMG-CoA reductase inhibitor is a statin drug.

4. The dosage unit of claim 1, wherein the insulin secretion stimulant is selected from the group consisting of glipizide, glimepiride and glyburide.

5. The dosage unit of claim 1, wherein the HMG-CoA reductase inhibitor is selected from the group consisting of simvastatin, atorvastatin calcium, fluvastatin sodium, lovastatin, pravastatin sodium, and rosuvastatin calcium.

6. The dosage unit of claim 1, wherein the insulin secretion stimulant is glipizide.

7. The dosage unit of claim 1, wherein the HMG-CoA reductase inhibitor is simvastatin.

8. The dosage unit of claim 1, wherein the insulin secretion stimulant is glipizide and the HMG-CoA reductase inhibitor is simvastatin.

9. The dosage unit of claim 1, wherein at least one of the insulin secretion stimulant and the HMG-CoA reductase inhibitor exhibits sustained-release characteristics.

10. A pharmaceutical dosage unit comprising about 5 to about 10 milligrams glipizide and about 20 to about 40 milligrams simvastatin.

11. The dosage unit of claim 10, wherein at least one of the glipizide and the simvastatin exhibits sustained-release characteristics.

12. A method of treating a diabetic patient comprising administering a pharmaceutical dosage unit, the dosage unit including a therapeutically effective amount of an insulin secretion stimulant in combination with a HMG-CoA reductase inhibitor.

13. The method of claim 12, wherein the insulin secretion stimulant is glipizide.

14. The method of claim 12, wherein the HMG-CoA reductase inhibitor is simvastatin.

15. The method of claim 12, wherein the insulin secretion stimulant is glipizide and the HMG-CoA reductase inhibitor is simvastatin.

16. The method of claim 12, wherein at least one of the insulin secretion stimulant and the HMG-CoA reductase inhibitor exhibits sustained-release characteristics.

17. A method for reducing the number of dosages administered to a diabetic patient by utilizing a combination of active agents, the method comprising the steps of:
a) combining in a single dosage unit a therapeutically effective amount of an insulin secretion stimulant and a HMG-CoA reductase inhibitor; and
b) administering to a diabetic patient the pharmaceutical dosage unit.

18. The method of claim 17, wherein the insulin secretion stimulant is glipizide.

19. The method of claim 17, wherein the HMG-CoA reductase inhibitor is simvastatin.

20. The method of claim 17, wherein the insulin secretion stimulant is glipizide and the HMG-CoA reductase inhibitor is simvastatin.

21. The method of claim 17, wherein at least one of the insulin secretion stimulant and the HMG-CoA reductase inhibitor exhibits sustained-release characteristics.

22. A method of treating a diabetic patient comprising administering to the patient a single dosage unit per day, the dosage unit including both a therapeutically effective amount of an insulin secretion stimulant exhibiting sustained-release characteristics, and a HMG-CoA reductase inhibitor.

23. The method of claim 22, wherein the insulin secretion stimulant is glipizide.

24. The method of claim 22, wherein the HMG-CoA reductase inhibitor is simvastatin.

25. The method of claim 22, wherein the insulin secretion stimulant is glipizide and the HMG-CoA reductase inhibitor is simvastatin.

26. A pharmaceutical dosage unit comprising a therapeutically effective amount of an antihyperglycemic biguanide compound in combination with a HMG-CoA reductase inhibitor.

27. The dosage unit of claim 26, wherein the antihyperglycemic biguanide compound is metformin hydrochloride.

28. The dosage unit of claim 26, wherein the HMG-CoA reductase inhibitor is a statin drug.

29. The dosage unit of claim 26, wherein the HMG-CoA reductase inhibitor is selected from the group consisting of simvastatin, atorvastatin calcium, fluvastatin sodium, lovastatin, pravastatin sodium, and rosuvastatin calcium.

30. The dosage unit of claim 26, wherein the HMG-CoA reductase inhibitor is simvastatin.

31. The dosage unit of claim 26, wherein the antihyperglycemic biguanide compound is metformin hydrochloride and the HMG-CoA reductase inhibitor is simvastatin.

32. The dosage unit of claim 26, wherein at least one of the antihyperglycemic biguanide compound and the HMG-CoA reductase inhibitor exhibits sustained-release characteristics.

33. A method of treating a diabetic patient comprising administering a pharmaceutical dosage unit, the dosage unit including a therapeutically effective amount of antihyperglycemic biguanide compound in combination with a HMG-CoA reductase inhibitor.

34. A method for reducing the number of dosages administered to a diabetic patient by utilizing a combination of active agents, the method comprising the steps of:
a) combining in a single dosage unit a therapeutically effective amount of an antihyperglycemic biguanide compound and a HMG-CoA reductase inhibitor; and
b) administering to a diabetic patient the pharmaceutical dosage unit.

35. A method of treating a diabetic patient comprising administering to the patient a single dosage unit per day, the dosage unit including both a therapeutically effective amount of an antihyperglycemic biguanide compound exhibiting sustained-release characteristics, and a HMG-CoA reductase inhibitor.
